(19) United States
(12) Patent Application Publication
(10) Pub. No.: US 2024/0268662 A1
(43) Pub. Date: Aug. 15, 2024

(54) OPHTHALMIC ILLUMINATION/OBSERVATION DEVICE

(71) Applicant: ARKADIY FARBEROV, Novato, CA (US)

(72) Inventor: ARKADIY FARBEROV, Novato, CA (US)

(21) Appl. No.: 18/107,791

(22) Filed: Feb. 9, 2023

(51) Int. Cl. A61B 3/117; A61B 3/125

(52) U.S. Cl. CPC A61B 3/1173; A61B 3/125

(57) ABSTRACT

An ophthalmic illumination/observation device for use in ophthalmology, which consists of a bi-concave contact lens with a front concave surface that matches an external surface of a patient's eye cornea onto which the front concave surface of the bi-concave contact lens is placed during the procedure, a rear concave surface on an opposite side of the bi-concave contact lens, and a separate optical gonio lens having a distal end and a proximal end with a convex surface on the proximal end that during the procedure is placed onto the rear concave surface of the bi-concave contact lens and matches the rear concave surface of the bi-concave contact lens. Preferably, the bi-concave contact lens is made from a soft elastic plastic material. The device provides efficiency of illumination and observation and is inexpensive to manufacture.

### FIELD OF THE INVENTION

The invention relates to the field of ophthalmic instruments, in particular, to ophthalmic illumination/observation devices. More specifically, the invention relates to an ophthalmic illumination/observation device for use in ophthalmology for observation of a human eye for diagnostics or during surgery with improved conditions for illumination and observation of a patient's eye.

### BACKGROUND OF THE INVENTION AND PRIOR ART

The anterior chamber of a human eye is commonly evaluated during slit lamp biomicroscopy, but the anterior chamber angle is hidden from ordinary view because of total internal reflection of light rays emanating from the angle structures. In other words, without gonioscopy, the additional diagnostic clues of disease are forever hidden from ordinary view. It requires additional efforts, skill and patient co-operation to view the normally concealed chamber angle by either indirect (angle structures viewed through a mirror) or direct (angle structures viewed directly) gonioscopic techniques. In other words, without gonioscopy, it is impossible to classify properties of such eye disease as, e.g., a glaucoma.

A gonioscopic lens or gonioscope is an instrument consisting of a contact lens to be fitted over the cornea of an eye and an optical system with which the interior of the eye can be viewed.

Heretofore, various gonioscopic devices have been known. The basic gonioscopic instrument used in the art is known as a Goldman “universal” lens and mirrors. After the initial development, the Goldman “universal” lens undergone many modifications which are known under various names, though the main principle remains unchanged. A concurrent gonioscope comprises an optical body with flat tapered sides having an entrance face which is flat or spherical, a spherical exit face which is applied to the cornea of the eye, and a reflecting face. The Goldman gonioscope is a universal three-reflection lens assembly for biomicroscopic investigation. With the help of lenses by Goldman in combination with a binocular microscope and a slit lamp or any other illumination device, minute variations of eye structures can be observed.

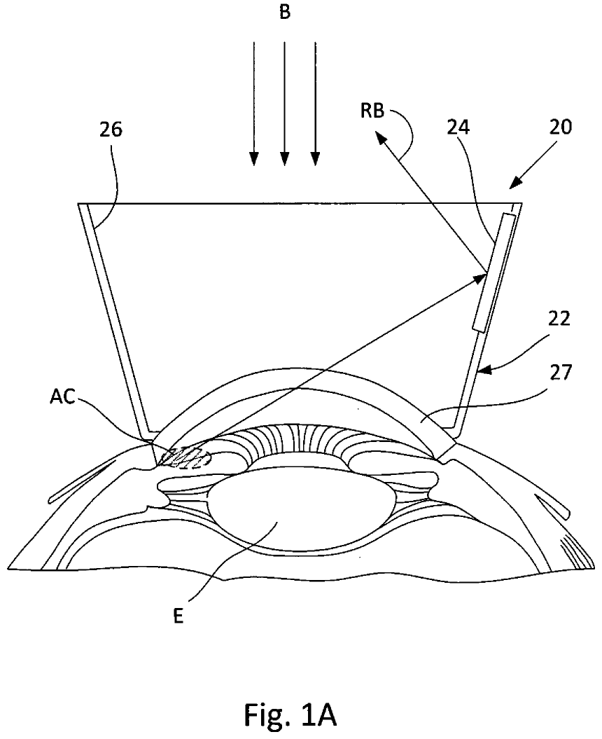

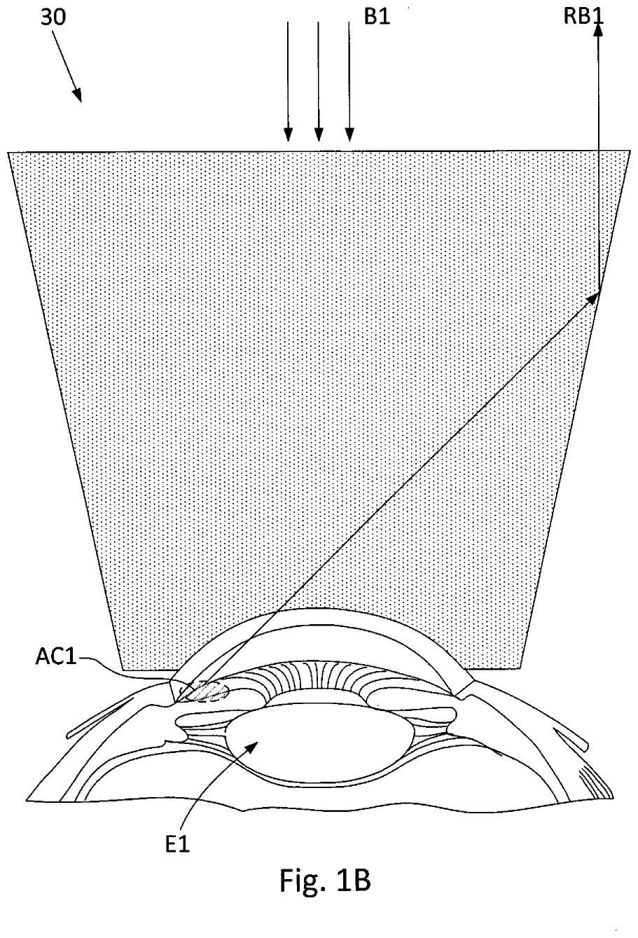

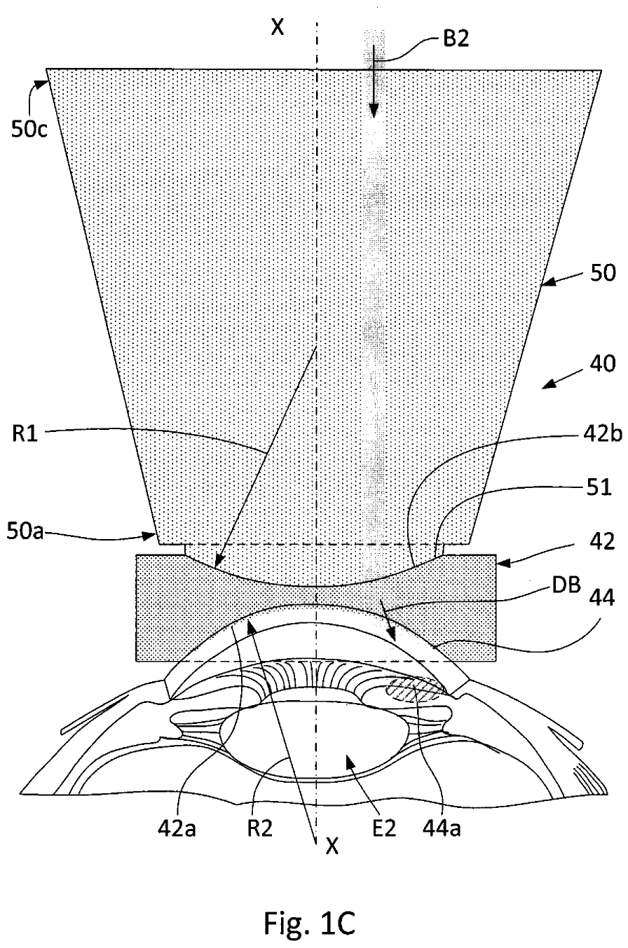

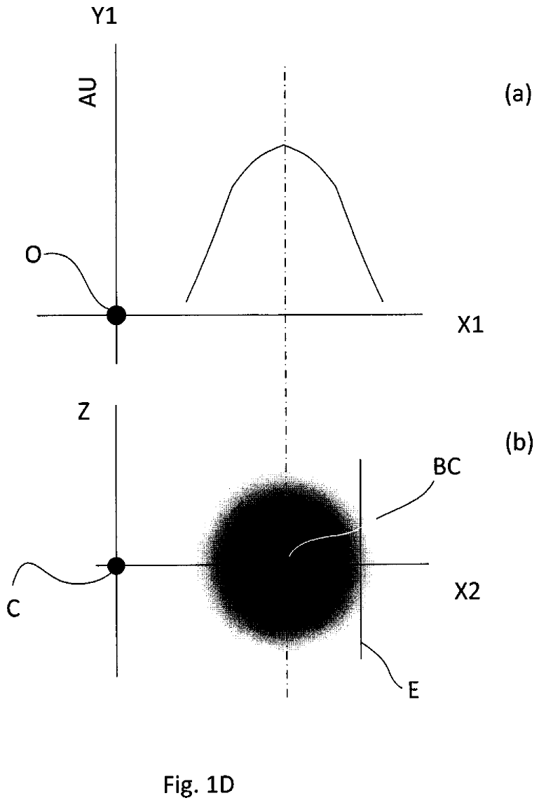

For example, known in the art is a gonioscope described in U.S. Pat. No. 6,942,343 issued on Sep. 13, 2005 to Arkadiy Farberov. This device is shown in a simplified form in FIG. 1A. The gonioscope, which is designated as an entity by reference numeral 20, has a hollow tapered body 22 with open top and bottom and with mirror surfaces 24 (although several such reflecting surfaces as the mirror 24 are used, only one of them is shown in FIG. 1A) formed on the inner side 26 of the gonioscope 20 or on the inserts placed into the recesses on the inner surface of the gonioscope. In operation, the lower open end of the gonioscope 20, which has a diameter that approximately matches the outer diameter of the patient's eye cornea, is placed onto the cornea 27, and a beam B of an illumination light emitted, e.g., from a slip lamp or any other light source (not shown), is directed onto the eye, while an observer, e.g., an ophthalmologist, observes the area of interest, e.g., an anterior angle chamber AC, in a scattered light RB. In other words, the area of interest is illuminated by a scattered light, i.e., without direct illumination by the incident beam B. As a result, in spite of the fact that the incident light is sufficiently bright, the image is not sufficiently contrast.

A conventional gonioscope of the aforementioned type is not necessarily hollow and may be comprised of a solid tapered body made of an optical material as shown in U.S. Pat. No. 4,664,490 filed by Lasag AG on May 2, 1985 and published on May 12, 1987. Such a solid gonioscope designated in general by reference numeral 30 is shown in a simplified form in FIG. 1B. The eye E1 is illuminated by a beam B1 emitted, e.g., from a slit lamp (not shown), and the area of interest AC1 is observed in a scattered light and seen by an observer via a reflected beam RB1.

Other examples of known gonioscopes are shown below.

US Patent Application Publication No. 20210030271 published on Feb. 4, 2021 (inventor A. Farberov) discloses a device that consists of a gonioscopic lens and gonioscopic lens support for supporting the lens during an ophthalmic procedure. The lens is a cylindrical body with tilted end faces on both end sides. The lens support is a part, which is placed with one side on the patient's eye, while the other side supports the gonioscopic lens. The eye support can be made in different modifications but, in any case, has a cavity capable of accommodating the eye cornea. The device has two optical lenses. The first lens is a convex lens formed on or attached to the proximal end face of the gonioscopic lens. The second lens is located at the distal end of the gonioscopic lens and may constitute either a concave lens formed on the gonioscopic lens eye support or a concave curvilinear surface of the cavity on the distal end of the gonioscopic lens.

U.S. Pat. No. 10,413,178 issue on Sep. 17, 2019 to R. Graham, at al. discloses a self-adhering, flexible gonioscopic lens for adhering to cornea and scleral regions of an eye. The device includes a central lens having a body including a contact surface and a viewing surface, the contact surface having a radius of curvature that approximates the radius of curvature of the cornea, an eye fixation system configured for fixing the central lens to the eye, wherein the eye fixation system is attached to the annular perimeter of the contact surface of the central lens, extending around only a portion of the annular perimeter of the contact surface of the central lens to define a cut-out portion in the eye fixation system of the gonioscopic lens.

Thus, one of the main goals of all types of gonioscopic optics is to improve illumination and observation of the anterior angle chamber of the eye being examined.

From an objective point of view, we have an outer convex spherical surface of the cornea of the eye. An ordinary gonioprism that is used for observing the eye normally has at its proximal end a concave sphere, the radius of which is approximately equal to the convex radius of the cornea. Due to the coincidence of the radii of the cornea and the gonio prism, optical contact is provided between the cornea and the prism. This spherical radius is normally equal to 8 mm.

In practice, however, the outer surface of the cornea is not always spherical and its radius is not always equal to 8 mm. In real life, the outer surface of the cornea is most often elliptical, and the difference in vertical and horizontal diameters reaches about 2 mm. In addition, this surface of the cornea is often astigmatic and aspherical. Furthermore, it has many local micro and macro irregularities.

These and many other variable features of the real human cornea with a constant sphericity of the gonio lenses makes the optical contact between them of a very poor quality. To reduce the influence of these factors in practice, an intermediate liquid is often used, which is introduced into the space between the cornea and the contact surface of the gonio lens. Such a liquid only partially compensates for the described real factors. Furthermore, the existing gonio lenses not always provide desired illumination and observation of the anterior angle chamber.

Thus, there is enough room for improvement in the field of ophthalmic illumination/observation devices.

### SUMMARY OF THE INVENTION

The invention relates to the field of ophthalmic instruments, in particular, to ophthalmic illumination/observation devices. More specifically, the invention relates to ophthalmic illumination/observation devices that improve conditions for observation of hard-to-observe areas such as an anterior chamber angle of a human eye, which normally is illuminated by a scattered light rather than a direct incident light.

An ophthalmic illumination/observation device of the invention (which hereinafter will be referred to as an ophthalmic illumination device, or merely as a device of the invention), consists of two separate or integrally connected parts, i.e., 1) a bi-concave contact lens having on one side a front concave surface that matches the shape of an external surface of a patient's eye cornea onto which the front concave surface of the bi-concave contact lens is placed during an ophthalmic procedure, and a rear concave surface on a side of the bi-concave contact lens opposite to the aforementioned one side; and 2) an optical gonio lens having a distal end and a proximal end with a convex surface on the proximal end that during the ophthalmic procedure is placed onto the rear concave surface of the bi-concave contact lens and that matches the rear concave surface of the bi-concave contact lens.

Preferably, the bi-concave contact lens is made from a soft elastic plastic material.

In an ophthalmic procedure, the bi-concave contact lens is placed on the patient's eye, and the bi-concave contact lens, which is made from a hard transparent optical material, is placed on the second concave surface of this contact lens, through which the inner surface of the eye is illuminated. Normal optical contact between these two spherical surfaces is ensured by the softness and elasticity of the contact lens material, which provides a normal optical contact between two surfaces.

A uniqueness of the device of the invention resides in the fact that the bi-concave contact lens has a specific geometry provides conditions for redirecting the incident beam and for illuminating the anterior angle chamber of the patient's eye with a direct light that penetrates deeper into the angle chamber than in the case of a conventional contact lens having a concave surface only on one side.

Silicon hydrogel soft elastic plastics such as Polymacon, Innofilcon or Lotraflicon, etc. is suitable for use as a material for the bi-concave contact lens. Due to their softness and elasticity, even with minimal pressure, such materials may completely repeat the micro and macro relief of the real cornea of the patient's eye and at the same time may provide high-quality optical contact between the cornea surface and the contact lens contact surface.

Another unique feature of the device of the invention is that the bi-concave contact lens is an assembly of two parts made from optical materials having different refractive indices, which are selected so that the incident illumination beam may be tilted closer to the zone of interest.

### BRIEF DESCRIPTION OF THE INVENTION

FIG. 1A is a simplified vertical sectional view of a conventional hollow multifaceted gonio lens with reflective mirrors on the inner wall.

FIG. 1B is a simplified vertical sectional view of a conventional monolithic gonio lens with reflection of the illumination beam from the side wall of the lens.

FIG. 1C is a vertical sectional view of the device of the invention consisting of an optical gonio lens and a bi-concave contact lens.

FIG. 1D illustrates change in the light intensity of the illumination spot from the center to the periphery and a digitized value of the light intensity.

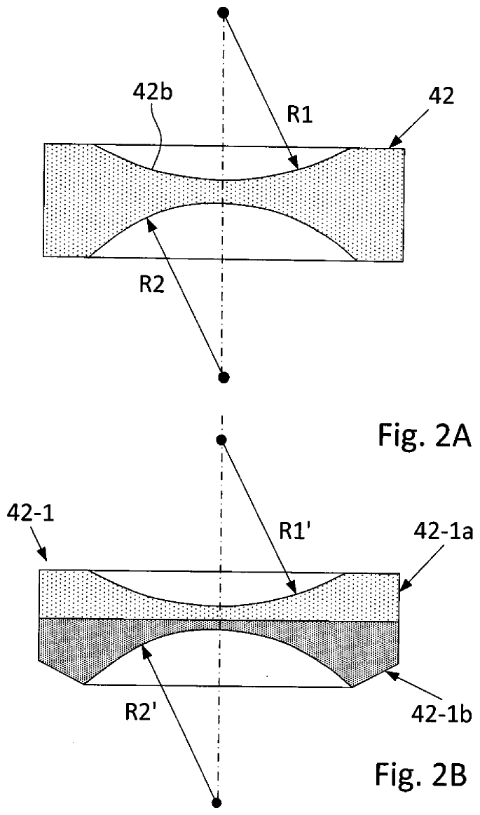

FIG. 2A is a cross-section of a monolithic bi-concave contact lens of the invention.

FIG. 2B is a cross-section of a composite bi-concave contact lens of the invention.

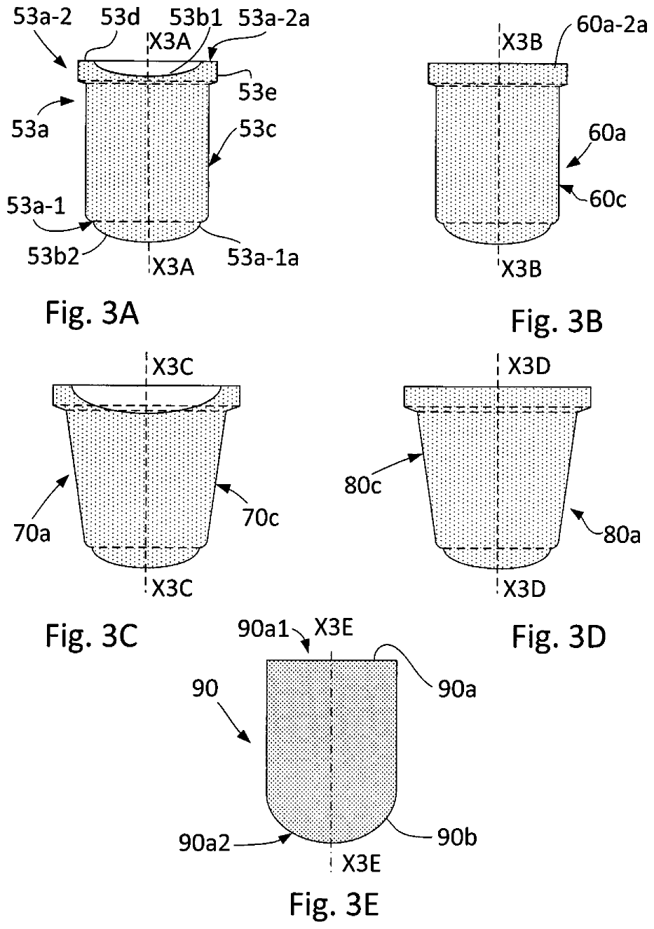

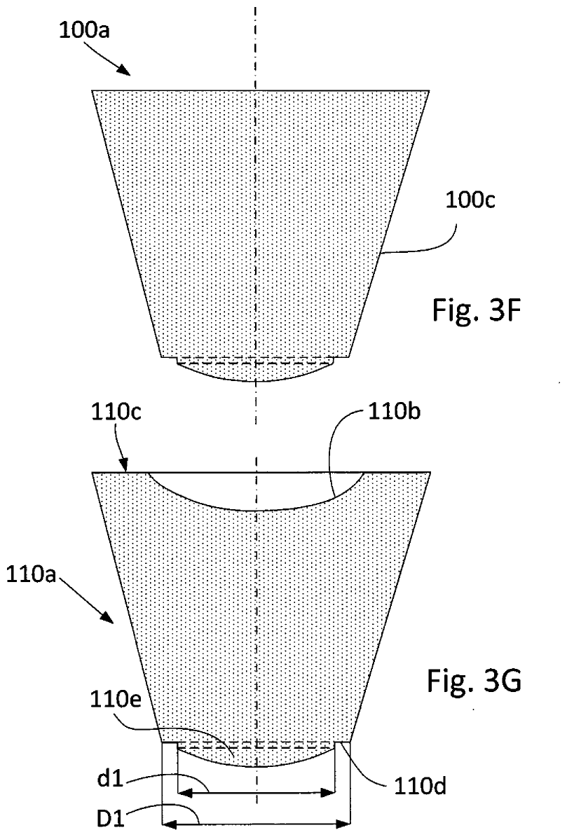

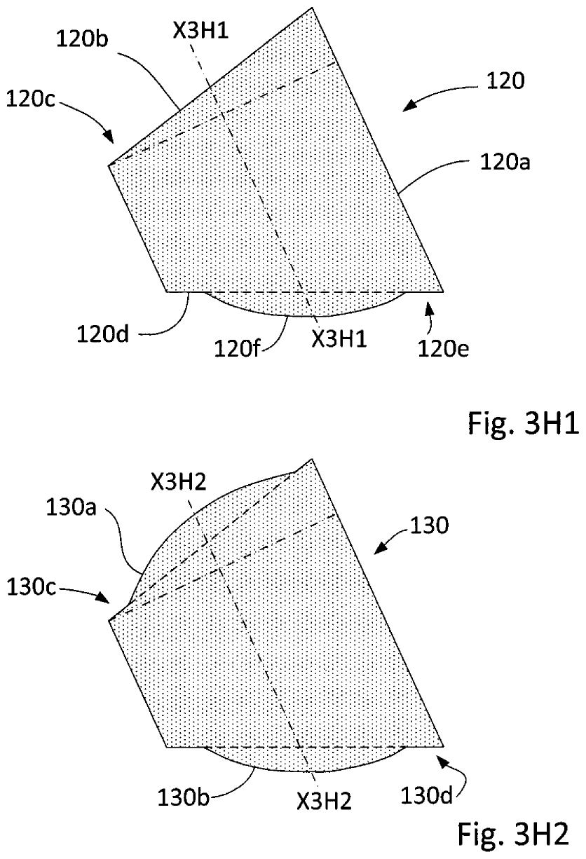

FIGS. 3A, 3B, 3C, 3D, 3E, 3F, 3G, 3H1, and 3H2 illustrate various modifications of the optical gonio lenses of the invention.

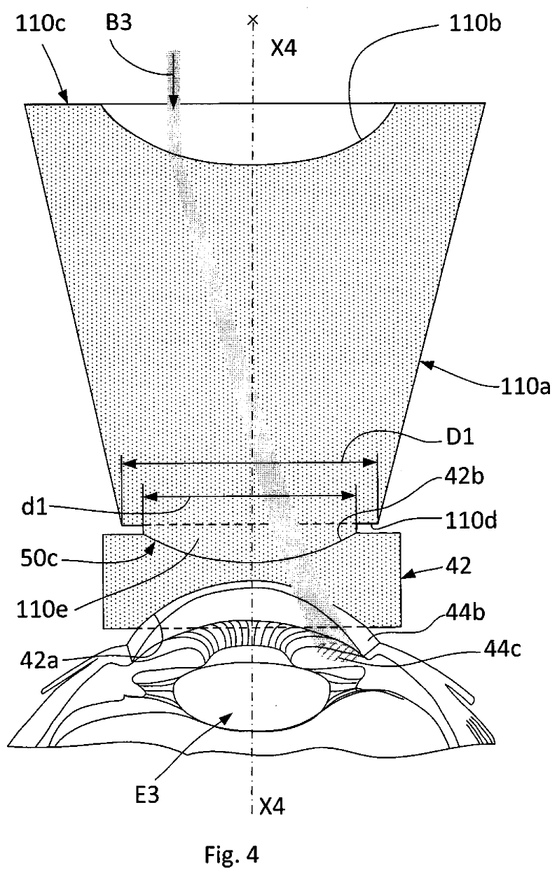

FIG. 4 shows a combination of the optical gonio lens of FIG. 3G with the bi-concave contact lens of FIG. 2A.

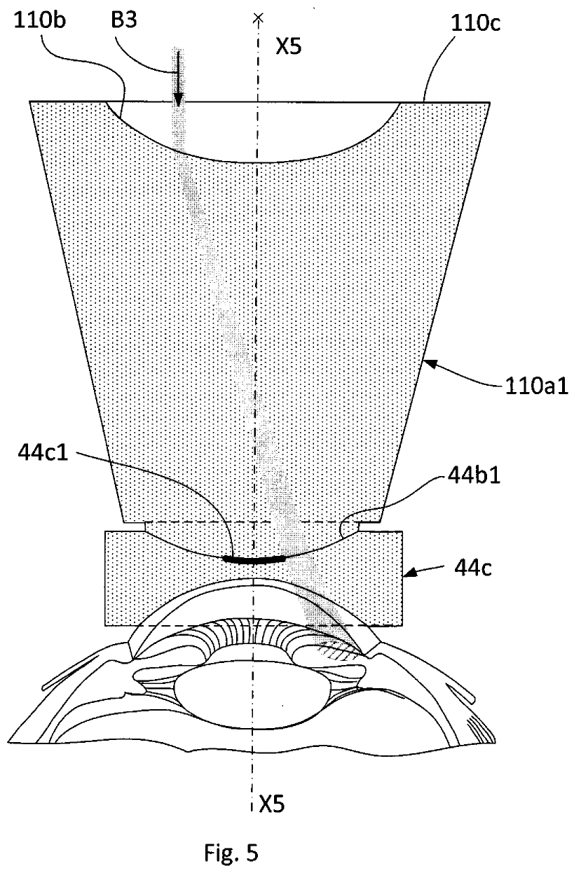

FIG. 5 shows a modification of the device of the invention, which is similar to one of FIG. 4, except that the rear concave surface of the bi-concave contact lens has a central light-impermeable portion.

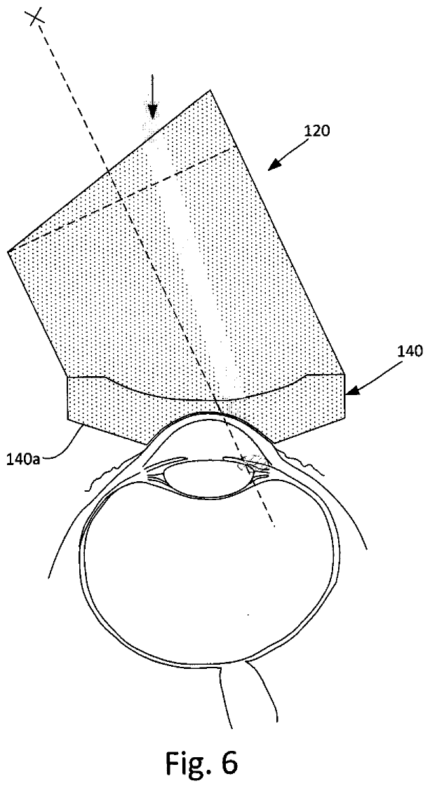

FIG. 6 shows a combination of the optical gonio lens of FIG. 3H1 with a bi-concave contact lens having a chamfered front end face.

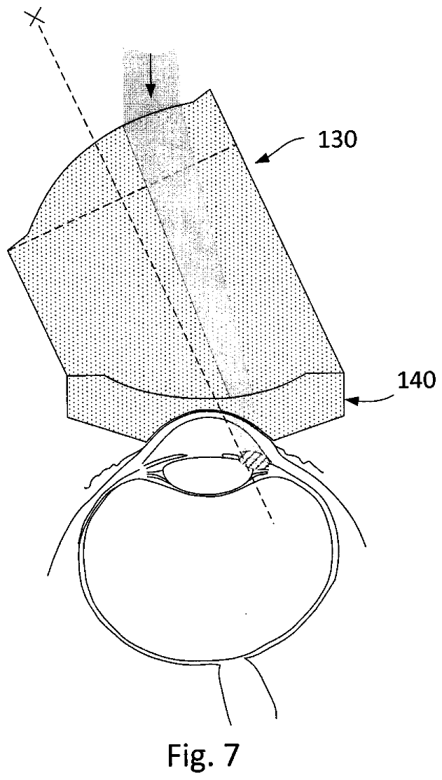

FIG. 7 shows a combination that is similar to one shown in FIG. 6 with the only difference that the be-concave contact lens of the modification shown in FIG. 3H2.

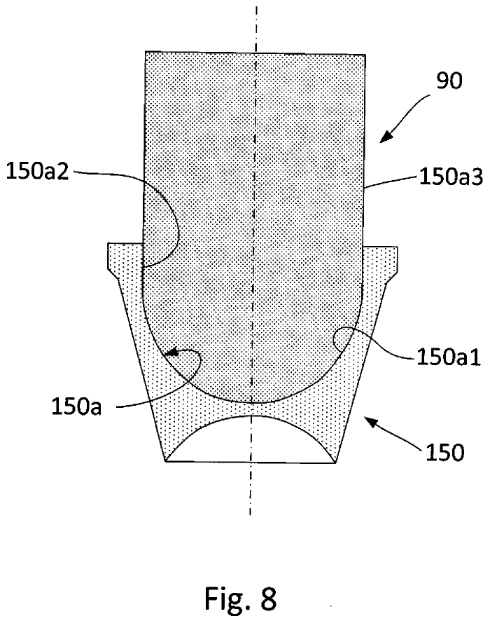

FIG. 8 illustrates a combination of the optical gonio lens of FIG. 3E with a bi-concave contact lens that has a conical cup-shaped configuration with an inner surface defined by a rear concave surface and a cylindrical inner surface extending in the direction opposite to the rear concave surface and matching the cylindrical shape of the optical gonio lens.

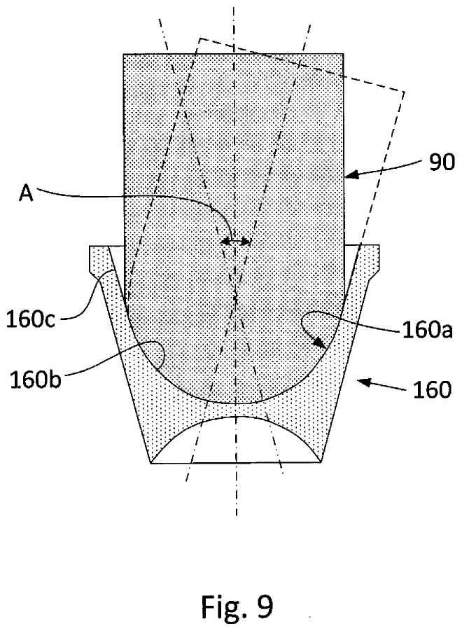

FIG. 9 illustrates a combination of the optical gonio lens of FIG. 3E with features that allow tilting of the optical gonio lens relative the bi-concave contact lens during the ophthalmic procedure.

### DETAILED DESCRIPTION OF PREFERRED EMBODIMENTS OF THE INVENTION

The invention relates to the field of ophthalmic instruments, in particular to ophthalmic illumination/observation devices. More specifically, the invention relates to ophthalmic illumination/observation devices for use in ophthalmology for observation of a human eye for diagnostics or during surgery with improved conditions for illumination and observation of a hard-to-observe anterior angle chamber.

In the context of the present patent specification, the term “proximal” relates to an end of the device or lens which is closer to an object being observed, and the term “distal” relates to an end that is closer to an observer.

As shown in FIG. 1C, an ophthalmic device of the invention designated in general by reference numeral 40 contains a bi-concave contact lens 42 having on one side a front concave surface 42a that matches a shape of an external surface of a patient's eye cornea 44 onto which the front concave surface 42a of the bi-concave contact lens 42 is placed during an ophthalmic procedure, and a rear concave surface 42b on a side of the bi-concave contact lens 42 opposite to the front concave surface 42a.

Another part that constitutes the device 40 is an optical gonio lens 50 that has a distal end 50c and a proximal end 50a with a convex surface 51 on the proximal end 50a that during the ophthalmic procedure is placed onto the rear concave surface 42b of the bi-concave contact lens 42 and that matches the rear concave surface 42b of the bi-concave contact lens 42.

According to one or several aspects of the invention, the bi-concave contact lens 42 is made preferably of a soft elastic, i.e., deformable transparent optical material. Examples of such materials are Silicon hydrogel soft elastic plastics such as Polymacon, Innofilcon or Lotraflicon, etc. Due to their softness and elasticity, even with minimal pressure, such materials may completely repeat the micro and macro relief of the real cornea 44 (FIG. 1C) of the patient's eye and at the same time may provide high-quality optical contact between the cornea 44 and the front concave surface 42a.

If necessary, the bi-concave contact lens 42 can be made of a non-deformable optical material.

The bi-concave contact lens 42 can be made disposable.

According to another aspect of the invention, the bi-concave contact lens 42 and the optical gonio lens 50 are made from optical materials of different refractive indices. These indices are selected so that, in combination with the rear concave surface 42b (FIG. 1C), it becomes possible to illuminate a hard-to-observe anterior angle chamber 44a of the patient's eye E2 by shifting the incident light beam DB (which is obtained after refraction of the initial illumination beam B2 that propagates from the light source (not shown) through the optical gonio lens, e.g., in the direction parallel to the longitudinal axis X-X of the device) closer to the anterior angle chamber 44a. It is understood that the incident light is refracted not only on the borders between the optical gonio lens and the bi-concave contact lens, but also on the border of the bi-concave contact lens with the patient's eye cornea. However, since the thickness of the eye cornea is insignificantly small as compared to the thicknesses of the lenses, the refraction on the cornea can be neglected.

In fact, the position and dimensions of the anterior angle chamber 44a depends not only on the refractory indices of the materials from which the bi-concave contact lens 42 and the optical gonio lens 50 are made, but also on the geometry of their curved surfaces, i.e., on the radius R1 of the rear concave surface 42b and the radius R2 of the front concave surface of the bi-concave contact lens 42.

In order to clarify a position of the direct-illumination light beam DB in the zone of interest, i.e., the hard-to-observe anterior angle chamber 44a (FIG. 1C), let us refer to FIG. 1D. This drawing illustrates change in the intensity of the illumination spot from the center of the spot to its periphery (image (b)) and a digitized value of the light intensity (image (a)). In the drawing, a black spot C designates a center of the pupil of the patient's eye when during the procedure the optical device 40 of the invention is applied onto the eye cornea 44. The black circle BC with gradual decrease of the black tone (in fact, the illuminated area is colored due to dispersion of the white-light components) from the center of the spot BC to the periphery is an image that illustrates decrease of the observed light spot density from the center of the spot BC to the periphery. Line E marks the most remote border of the area of interest 44a. In FIG. 1D (a), the abscissa axis OX1 is used for plotting the spot dimension in the diametrical direction CX2, and the ordinate axis OY1 on the upper chart (b) shows a digitized value of the light intensity of the light incident on the area of interest 44a (FIG. 1C). Axis OY1 passes vertically to the plane of the drawings via black spot C of FIG. 1D(a).

A separate view of the bi-concave contact lens 42 of FIG. 1C is shown in FIG. 2A. This lens has a monolithic structure and is molded from a material of a predetermined refractory index different from one of the optical gonio lens 50.

However, in order to shift the incident illumination beam DB further to the anterior angle chamber 44a (FIG. 1C) of the investigated eye, the bi-concave lens 42 may have a composite structure, i.e., may have a form of a doublet 42-1 of the type illustrated in FIG. 2B. This lens is assembled from an upper lens portion 42-1a and a lower lens portion 42-1b secured together through a thin layer of an optical glue (not shown) and are made from optical materials of different refractive indices. Furthermore, by properly selecting the designs and materials of the upper lens portion 42-1a and a lower lens portion 42-1b, an achromatic doublet may to some extent suppress aberration.

According to the invention, the optical gonio lens also may have different geometrical configurations with a variety of shapes and arrangements that are selected to adjust dimensions, intensity of illumination light, and positions of the light spot in the zone of interest. The geometrical shapes are selected also with reference to the cost and disposability of both the optical gonio lens, the bi-concave contact lens, or the entire devices. The possible geometrical configurations of the optical gonio lenses are shown in FIGS. 3A, 3B, 3C, and 3D.

In general, each optical gonio lens has a proximal end, a rear end opposite to the proximal end, and an intermediate portion between the proximal end and the rear end, wherein the intermediate portion may have a cylindrical shape, conical circular shape, a conical multiple-faced shape, or a combination of the above. In the modifications of FIGS. 3A, 3B, 3C, 3D, and 3E, the intermediate portion is always symmetrical and has an axis of symmetry and longitudinal axis that coincides with the axis of symmetry.

FIG. 3A illustrates an optical gonio lens 53a of the invention that has a cylindrical intermediate portion 53c, a proximal end 53a-1, a front end face 53a-1a on the proximal end 53a-1, a distal end 53a-2, and a rear end face 53d on the distal end 53a-2. In the modification of FIG. 3A, the optical gonio lens has a flange 53e on the distal end 53a-2, a concave optical lens 53b1 on the rear end face 53d, and a convex lens 53b2 formed on the front end face 53e-1a. For convenience of holding, the flange 50e is greater than the diameter of the rear end face 53a-2a. A diameter of the convex optical lens 53b2 is smaller than the diameter of the cylindrical intermediate portion 50c1.

FIG. 3B illustrates an optical gonio lens 60a of the invention that is similar to shown in FIG. 3A, except that the rear end face 60a-2a is flat, does not have a concave lens, and is perpendicular to the longitudinal axis X3B-X3B of the cylindrical intermediate portion 60c. X3B-X3B designates a longitudinal axis of the optical gonio lens 60a.

A modification of an optical gonio lens 70a shown in FIG. 3C is similar to one shown in FIG. 3A, except that the intermediate portion 70c has a conical shape. X3C-X3C designates a longitudinal axis of the optical gonio lens 70a.

A modification of a gonio lens 80a of FIG. 3D is similar to one shown in FIG. 3B, except that the intermediate portion 80c has a conical shape. X3D-X3D designates a longitudinal axis of the optical gonio lens 80a.

In a modification of FIG. 3E, the optical gonio lens is entirely cylindrical with a flat rear end face 90a on the distal end 90a1 and a convex lens 90b on the proximal end 90a2. Such a configuration is inexpensive to manufacture and may be disposable.

A modification of an optical gonio lens 100a shown in FIG. 3F is similar to one shown in FIG. 3A, except that the entire intermediate portion 70c has a conical shape.

A modification of an optical gonio lens 110a shown in FIG. 3G is similar to one shown in FIG. 3F, except that a concave lens 110b is made on the flat rear end face 110c of the conical body. In this modification of the optical gonio lens 110a (as well as in other modifications, except for one in FIG. 3E), the diameter d1 of the convex lens 110e on the front end face 110d is smaller than the diameter od the end face 110d.

In a modification of FIG. 3H1, an optical gonio lens 120 has a cylindrical intermediation portion 120a with an axial line X3H-X3H of the cylindrical portion. A flat rear end face 120b on the distal end 120c of the optical gonio lens 120 is inclined relative to the axial line X3H-X3H, a front end face 120d that is formed on the proximal end 120e is inclined relative to the axial line X3H-X3H so that the intermediate portion 120a becomes truncated, and a concave lens 120f is formed on the front end face 120d. An optical gonio lens 130 of FIG. 3H2 is similar to one shown in FIG. 3H and differs from the modification of FIG. 3H by having convex lenses 130a and 130b on the rear and front end faces 130c and 130d, respectively.

The modifications of FIGS. 3H1 and 3H2 provide more convenient observation of the area of interest.

Having described various modifications of the optical gonio lenses and bi-concave contact lenses separately, let us consider their mutual positions and interaction when they are brought in contact with each other in the course of ophthalmic procedures.

FIG. 4 shows combination of the optical gonio lens 110A of FIG. 3G with the bi-concave contact lens 42 of FIG. 2A. Other designations are the same as in FIG. 1C, FIG. 2A, and FIG. 3G. For use in the ophthalmic procedure, the bi-concave contact lens 42 is applied to the cornea 44b of the patient's eye E3. If necessary, a small amount of an appropriate lubricant (not shown) is placed in the area of contact of bi-concave lens 42 and the cornea 44b. The convex lens 110e of the optical gonio lens 110a is brought in contact with the rear contact surface 42b of the bi-concave contact lens 42, and a light beam B3 is directed to the zone of interest, in this case, to the anterior angle chamber 44c of the eye E3. The beam B3 penetrates through the surface of the concave lens 110b to the optical gonio lens 110a and, since the optical material of the bi-concave contact lens 42 ( [symbol] [symbol] [symbol] [symbol] [symbol] [symbol] [symbol] [symbol] ?) differs from that of the optical gonio lens 110a, is refracted and tilted further towards the zone of interest 44c.

Since the optical gonio lens 110a has a concave lens 110b formed on its distal end face 110c, in the combination of FIG. 4 the beam B3 will diverge, and this diversion will expand the size of the light spot that illuminates the zone of interest. Thus, the combination of FIG. 4 is suitable for situation when it is necessary to increase the size of the illumination spot. It is understood that in this case the brightness of the illumination spot will be decreased.

In the modification of FIG. 5, the situation is the same as in the case of FIG. 4, except that the rear concave surface 44b1 of the bi-concave contact lens 42c has a central light-impermeable portion 44c1. Other designations are the same as in FIG. 4. The combination of FIG. 5 provides an annular shape (not shown) of the illumination areas.

FIG. 6 shows a combination of the optical gonio lens 120 presented in FIG. 3H1 with a bi-concave contact lens 140. The bi-concave contact lens of this modification differs from those of the previous modifications in that its end face 140a is chamfered and constitutes a conical surface.

FIG. 7 shows a combination that is similar to one shown in FIG. 6 with the only difference that the be-concave contact lens 140 is combined with the optical gonio lens 130 of modification shown in FIG. 3H2.

FIG. 8 illustrates a combination of the optical gonio lens 90 of FIG. 3E with the bi-concave contact lens 150. The bi-concave contact lens 150 has a conical cup-shaped configuration with an inner surface 150a defined by a rear concave surface 150a1 and a cylindrical inner surface 150a2 extending in the direction opposite to the rear concave surface and matching the cylindrical shape 150a3 of the optical gonio lens 90.

FIG. 9 illustrates a combination of the optical gonio lens 90 of FIG. 3E with the bi-concave contact lens 160. The bi-concave contact lens 90 has a conical cup-shaped configuration with an inner surface 60a defined by a rear concave surface 160b and an inner conical extension 160c diverging in the direction opposite to the rear concave surface 160b and serving to limit an angle A of inclination of the optical gonio lens 90 relative the bi-concave contact lens 160 during the ophthalmic procedure. A broken-line image shown the optical gonio lens 90 in a tilted position.

The device of the invention was shown and described in different specific modifications. However, the invention is not limited to these modifications which were shown only as examples, and various changes and ramifications are possible within the scope of the attached patent claims. For example, biologically acceptable resilient soft optical materials other than those mentioned in the specification may be used for manufacturing the bi-concave contact lens. Furthermore, other optical materials may be used for the solid optical gonio lenses. The multiple-faceted hollow gonio lenses have the same geometry and dimensions as conventional gonio lenses, except that they must have a convex lens on the front end face.

1. An ophthalmic illumination/observation device comprising:
a bi-concave contact lens having on one side a front concave surface that matches a shape of an external surface of a patient's eye cornea onto which the front concave surface of the bi-concave contact lens is placed during an ophthalmic procedure, and a rear concave surface on a side of the bi-concave contact lens opposite to said one side; and
an optical gonio lens having a distal end and a proximal end with a convex surface on the proximal end that during the ophthalmic procedure is placed onto the rear concave surface of the bi-concave contact lens and that matches the rear concave surface of the bi-concave contact lens.

2. The ophthalmic illumination/observation device of claim 1, wherein the bi-concave contact lens is made of an optical material selected from the group consisting of a non-deformable transparent optical material and a deformable transparent optical material.

3. The ophthalmic illumination/observation device of claim 2, wherein the bi-concave contact lens is disposable.

4. The ophthalmic illumination/observation device of claim 2, wherein the bi-concave contact lens and the optical gonio lens are made from optical materials of different refractive indices and different optical dispersion for tilting an incident beam closer to an area of interest.

5. The ophthalmic illumination/observation device of claim 3, wherein the bi-concave contact lens and the optical gonio lens are made from optical materials of different refractive indices and different optical dispersion for tilting an incident beam closer to an area of interest.

6. The ophthalmic illumination/observation device of claim 2, wherein the optical gonio lens comprises said proximal end, a distal end on a side opposite to the proximal end, a rear end face on the distal end, a front end face on the proximal end, and an intermediate portion between the proximal end and the distal end, wherein the intermediate portion has a shape selected from the group consisting of a cylindrical shape, conical shape, a conical multiple-facet shape, a cylindrical truncated shape, and a combination of the above, wherein said intermediate portion has a symmetry, an axis of symmetry, and a first longitudinal axis that coincides with the axis of symmetry.

7. The ophthalmic illumination/observation device of claim 6, wherein the rear end face is flat.

8. The ophthalmic illumination/observation device of claim 6, wherein the convex surface on the proximal end of the optical gonio lens comprises a first convex optical lens.

9. The ophthalmic illumination/observation device of claim 8, further comprising a first concave optical lens on the rear end face.

10. The ophthalmic illumination/observation device of claim 6, wherein the bi-concave contact lens has a conical cup-shaped configuration with an inner surface defined by said rear concave surface and an inner conical extension diverging in the direction opposite to the rear concave surface and serving to limit an angle of inclination of the optical gonio lens relative the bi-concave contact lens during the ophthalmic procedure.

11. The ophthalmic illumination/observation device of claim 6, wherein the bi-concave contact lens has a conical cup-shaped configuration with an inner surface defined by said rear concave surface and an inner surface extending in the direction opposite to the rear concave surface and matching the cylindrical shape of the optical gonio lens.

12. The ophthalmic illumination/observation device of claim 6, wherein the rear concave surface of the bi-concave contact lens has a central light-impermeable portion.

13. The ophthalmic illumination/observation device of claim 6, wherein in case of said conical multiple-facet shape, the optical gonio lens diverges in a direction opposite to the front concave surface and has reflective mirrors on at least a part of said multiple facet shape.

14. The ophthalmic illumination/observation device of claim 7, wherein the a bi-concave contact lens has a second longitudinal axis that during the ophthalmic procedure coincides with an optical axis of a patient's eye, the first longitudinal axis may coincide with the second optical axis or be inclined relative to the second longitudinal axis during the ophthalmic procedure, and wherein the rear end face of the optical gonio lens is perpendicular to the first longitudinal axis.

15. The ophthalmic illumination/observation device of claim 7, wherein the a bi-concave contact lens has a second longitudinal axis that during the ophthalmic procedure coincides with an optical axis of a patient's eye, the first longitudinal axis may coincide with the second optical axis or be inclined relative to the second longitudinal axis during the ophthalmic procedure, and wherein the rear end face of the optical gonio lens is inclined relative to the first longitudinal axis.

16. The ophthalmic illumination/observation device of claim 6, further comprising a first convex lens on the front end face, wherein the front end face has a first diameter, and the convex lens has a second diameter that is smaller than the first diameter.

17. The ophthalmic illumination/observation device of claim 16, further comprising a second convex lens on the rear end face.

18. The ophthalmic illumination/observation device of claim 6, wherein the rear end face on the distal end of the optical gonio lens has a diameter and a flange that for convenience of holding is larger than the diameter of the distal end of the optical gonio lens.

19. The ophthalmic illumination/observation device of claim 7, wherein the rear end face on the distal end of the optical gonio lens has a diameter and a flange that for convenience of holding is larger than the diameter of the distal end of the optical gonio lens.
